(19) United States
(12) Patent Application Publication
(10) Pub. No.: US 2026/0224385 A1
(43) Pub. Date: Aug. 6, 2026

(54) Lumbosacral Orthosis (LSO) and Method of Using Same

(71) Applicant: Motif Medical, LLC, Asheville, NC (US)

(72) Inventor: Eric Robert Peterson, Raleigh, NC (US)

(73) Assignee: Motif Medical, LLC, Asheville, NC (US)

(21) Appl. No.: 19/426,958

(22) Filed: Dec. 19, 2025

Related U.S. Application Data
(60) Provisional application No. 63/752,273, filed on Jan. 31, 2025.

(51) Int. Cl. A61F 5/02; A61F 5/32; A61F 7/00; A61F 7/02

(52) U.S. Cl. CPC A61F 5/028; A61F 5/32; A61F 2007/005; A61F 7/02; A61F 2007/0228

(57) ABSTRACT

A lumbar support system. The lumbar support system may include an orthotic support band, which may include a central portion; a first side portion extending from a first side of the central portion; a second side portion extending from a second side of the central portion; a first connector portion disposed on either of an inner or outer surface of either of the first or second side portion and a second connector portion disposed on the other of the inner or outer surface of the other of the first or second side portion, the first and second connector portion may be configured to engage with one another to secure the first and second side portion together; and an adjustable pull strap configured to allow for adjusting the orthotic support band about a user. The system may also include a posterior support panel, anterior support panel, and/or a pelvic strap.

### RELATED APPLICATIONS

This application is related and claims priority to U.S. Provisional Patent Application No. 63/752,273, filed on Jan. 31, 2025, the application of which is incorporate herein by reference in its entirety.

### TECHNICAL FIELD

The subject matter relates generally to a support belt, and more particularly to a lumbosacral orthosis (LSO) and method of using.

### BACKGROUND

Lower back pain and pelvic pain are incredibly common among pregnant women, as the body's center of gravity shifts to accommodate a growing belly. Pregnancy back braces currently exist that are used to alleviate the strain put on abdominal muscles, lower back, hips and the pelvic floor as pregnancy progresses, relieving the aches and pains that go along with it. Pregnancy back braces can also help improve circulation, especially in the second and third trimester. As a baby bump grows, a pregnancy back brace can help lift the weight off of the blood vessels that flow to the lower extremities, promoting good circulation and reducing fatigue in the legs and feet. However, current pregnancy back braces are limited with respect to, for example, their effectiveness for relieving pregnancy-related pressure from the abdomen, hips, and lower back.

### SUMMARY

In one embodiment, a lumbar support system is provided. The lumbar support system may include an orthotic support band. The orthotic support band may include a central portion; a first side portion extending from a first side of the central portion; a second side portion extending from a second side of the central portion; a first connector portion disposed on either of an inner surface or an outer surface of an end portion of either of the first side portion or the second side portion and a second connector portion disposed on the other of the inner surface or the outer surface of an end portion of the other of the first side portion or the second side portion, wherein the first connector portion and the second connector portion may be configured to engage with one another to secure the first side portion and the second side portion together; and an adjustable pull strap attached to the orthotic support band, and configured to allow for adjusting a fit of the orthotic support band about a user. The lumbar support system may further include one or more of a posterior support panel, anterior support panel, and/or a pelvic strap. The central portion may further include a posterior support panel attachment, wherein the posterior support panel attachment may be configured to secure the posterior support panel at the central portion. The posterior support panel attachment may include a pouch configured to receive the posterior support panel therein. The pouch may be disposed on either of an exterior or interior surface of the central portion of the orthotic support band. The anterior support panel may be configured to be attachable to either of the first side portion or the second side portion of the orthotic support band. The anterior support panel may be configured to be attachable to an inside surface of either of the first side portion or the second side portion, such that when the orthotic support band is attached and secured about the user the anterior support panel is directly adjacent an anterior portion of the user. The lumbar support system of claim 1, wherein the pelvic strap is configured to extend from the central portion of the orthotic support band to either of the first side portion or the second side portion of the orthotic support band when the first side portion and the second side portion are secured together. The pelvic strap may include a posterior end having a first attachment mechanism disposed thereon and an anterior end having one or more finger straps extending therefrom, and wherein the one or more finger straps each may include a second attachment mechanism at their distal ends. The posterior end of the pelvic strap may be configured to be attachable to an inside surface of the central portion via the first attachment mechanism, and the anterior end of the pelvic strap may be configured to be attachable to an outside surface of either of the first side portion or the second side portion via the second attachment mechanism. The lumbar support system my further include a cold/hot pack holder, wherein the cold/hot pack holder may be configured to attach to the pelvic strap at about a mid-point thereof, and may be further configured to retain a cold/hot pack. The adjustable pull strap may include a first portion and a second portion, wherein the first portion may extend from about a middle point of the central portion in a first direction and the second portion may extend from about a middle point of the central portion in a second direction opposite of the first direction, and wherein an end portion of either or both of the first portion and second portion may be configured to releasably attach to an outer surface of the first side portion or second side portion.

In another embodiment, a method of using a lumbar support system is provided. The method may include providing an orthotic support band. The orthotic support band may include a central portion; a first side portion extending from a first side of the central portion; a second side portion extending from a second side of the central portion; a first connector portion disposed on either of an inner surface or an outer surface of an end portion of either of the first side portion or the second side portion and a second connector portion disposed on the other of the inner surface or the outer surface of an end portion of the other of the first side portion or the second side portion, wherein the first connector portion and the second connector portion may be configured to engage with one another to secure the first side portion and the second side portion together; and an adjustable pull strap attached to the orthotic support band, and configured to allow for adjusting a fit of the orthotic support band about a user. The lumbar support system may further include one or more of a posterior support panel, anterior support panel, and/or a pelvic strap. The method may further include providing one or more of a posterior support panel, anterior support panel, and/or a pelvic strap; optionally, attaching one or both of the posterior support panel and the anterior support panel to the orthotic support band; positioning and securing the orthotic support band about a user's body; optionally, adjusting the adjustable pull strap to a desired fit about the user's body; and optionally, attaching the pelvic strap to the orthotic support band. The method may further include attaching a cold/hot pack holder to the pelvic strap, and attaching or inserting a cold/hot pack to or in the cold/hot pack holder. The central portion of the orthotic support band may further include a posterior support panel attachment, wherein the posterior support panel attachment may be configured to secure the posterior support panel at the central portion of the orthotic support band. The posterior support panel attachment may include a pouch configured to receive the posterior support panel therein. The pouch may be disposed on either of an exterior or interior surface of the central portion of the orthotic support band. The anterior support panel may be configured to be attachable to either of the first side portion or the second side portion of the orthotic support band. The anterior support panel may be configured to be attachable to an inside surface of either of the first side portion or the second side portion of the orthotic support band, such that when the orthotic support band is attached and secured about the user the anterior support panel may be directly adjacent an anterior portion of the user. The pelvic strap may be configured to extend from the central portion of the orthotic support band to either of the first side portion or the second side portion of the orthotic support band when the first side portion and the second side portion are secured together. The pelvic strap may include a posterior end having a first attachment mechanism disposed thereon and an anterior end having one or more finger straps extending therefrom, and wherein the one or more finger straps each may include a second attachment mechanism at their distal ends. The posterior end of the pelvic strap may be configured to be attachable to an inside surface of the central portion of the orthotic support band via the first attachment mechanism, and the anterior end of the pelvic strap may be configured to be attachable to an outside surface of either of the first side portion or the second side portion of the orthotic support band via the second attachment mechanism. The cold/hot pack holder may be configured to attach to the pelvic strap at about a mid-point thereof, and may be further configured to retain a cold/hot pack.

### BRIEF DESCRIPTION OF THE DRAWINGS

Having thus described the subject matter in general terms, reference will now be made to the accompanying drawings, which are not necessarily drawn to scale, and wherein:

FIG. 1 and FIG. 2 illustrate a front (or inside) perspective view and a back (or outside) perspective view, respectively, of an example LSO in an expanded state, in accordance with an embodiment of the invention;

FIG. 3 illustrates a perspective view of an example LSO in an as-worn state, in accordance with an embodiment of the invention;

FIG. 4 and FIG. 5 illustrate a front plan view and a back plan view, respectively, of an example LSO in an expanded state, in accordance with an embodiment of the invention;

FIG. 6A, FIG. 6B, FIG. 6C, and FIG. 6D illustrate a first end view, a second end view, a first side view, and a second side view, respectively, of an example LSO in an expanded state, in accordance with an embodiment of the invention;

FIG. 7 and FIG. 8 illustrate a front perspective view and a back perspective view, respectively, of an example LSO in an expanded state and including a postpartum (or pelvic) strap, in accordance with an embodiment of the invention;

FIG. 9 illustrates a perspective view of an example LSO in an as-worn state and including a postpartum (or pelvic) strap, in accordance with an embodiment of the invention;

FIG. 10 illustrates a perspective view of an example postpartum (or pelvic) strap of the LSO, in accordance with an embodiment of the invention;

FIG. 11A, FIG. 11B, FIG. 11C, and FIG. 11D illustrate a front plan view, a back plan view, a side view, and an end view, respectively, of the postpartum (or pelvic) strap shown in FIG. 10, in accordance with an embodiment of the invention;

FIG. 12 and FIG. 13 illustrate a front perspective view and a back perspective view, respectively, of an example LSO in an expanded state and including both a postpartum (or pelvic) strap and a cold/hot pack, in accordance with an embodiment of the invention;

FIG. 14 illustrates a perspective view of an example LSO in an as-worn state and including both a postpartum (or pelvic) strap and a cold/hot pack, in accordance with an embodiment of the invention;

FIG. 15 illustrates a perspective view of an example of a cold/hot pack of the LSO, in accordance with an embodiment of the invention;

FIG. 16A and FIG. 16B illustrate a front perspective view and a back perspective view, respectively, of an example cold/hot pack pocket of the LSO, in accordance with an embodiment of the invention;

FIG. 17A, FIG. 17B, and FIG. 17C illustrate a front view, a side view, and an end view, respectively, of the cold/hot pack shown in FIG. 15, in accordance with an embodiment of the invention;

FIG. 18A, FIG. 18B, FIG. 18C, and FIG. 18D illustrate a front view, a back view, a side view, and an end view, respectively, of the cold/hot pack pocket shown in FIG. 16A and FIG. 16B, in accordance with an embodiment of the invention;

FIG. 19 and FIG. 20 illustrate a front perspective view and a back perspective view, respectively, of an example LSO in an expanded state and including an example of a rectangle-shaped anterior panel, in accordance with an embodiment of the invention;

FIG. 21 illustrates a perspective view of an example LSO in an as-worn state and including an example of a rectangle-shaped anterior panel, in accordance with an embodiment of the invention;

FIG. 22 illustrates a perspective view of an example rectangle-shaped anterior panel of the LSO, in accordance with an embodiment of the invention;

FIG. 23A, FIG. 23B, FIG. 23C, and FIG. 23D illustrate a front view, a back view, a side view, and an end view, respectively, of the rectangle-shaped anterior panel shown in FIG. 22, in accordance with an embodiment of the invention;

FIG. 24 and FIG. 25 illustrate a front perspective view and a back perspective view, respectively, of an example LSO in an expanded state and including an example of an oval-shaped anterior panel, in accordance with an embodiment of the invention;

FIG. 26 illustrates a perspective view of an example LSO in an as-worn state and including an example of an oval-shaped anterior panel, in accordance with an embodiment of the invention;

FIG. 27 illustrates a perspective view of an example oval-shaped anterior panel of the LSO, in accordance with an embodiment of the invention;

FIG. 28A, FIG. 28B, FIG. 28C, and FIG. 28D illustrate a front view, a back view, a side view, and an end view, respectively, of the oval-shaped anterior panel shown in FIG. 27, in accordance with an embodiment of the invention;

FIG. 29 and FIG. 30 illustrate a front perspective view and a back perspective view, respectively, of an example LSO in an expanded state and including both a postpartum (or pelvic) strap and a rectangle-shaped anterior panel, in accordance with an embodiment of the invention;

FIG. 31 illustrates a perspective view of an example LSO in an as-worn state and including both a postpartum (or pelvic) strap the rectangle-shaped anterior panel, in accordance with an embodiment of the invention;

FIG. 32 and FIG. 33 illustrate a front perspective view and a back perspective view, respectively, of an example LSO in an expanded state and including a postpartum (or pelvic) strap, a cold/hot pack, and a rectangle-shaped anterior panel, in accordance with an embodiment of the invention;

FIG. 34 illustrates a perspective view of an example LSO in an as-worn state and including a postpartum (or pelvic) strap, a cold/hot pack, and a rectangle-shaped anterior panel, in accordance with an embodiment of the invention;

FIG. 35 and FIG. 36 illustrate a front perspective view and a back perspective view, respectively, of an example LSO in an expanded state and including both a postpartum (or pelvic) strap and an oval-shaped anterior panel, in accordance with an embodiment of the invention;

FIG. 37 illustrates a perspective view of an example LSO in an as-worn state and including both a postpartum (or pelvic) strap the oval-shaped anterior panel, in accordance with an embodiment of the invention;

FIG. 38 and FIG. 39 illustrate a front perspective view and a back perspective view, respectively, of an example LSO in an expanded state and including a postpartum (or pelvic) strap, a cold/hot pack, and an oval-shaped anterior panel, in accordance with an embodiment of the invention;

FIG. 40 illustrates a perspective view of an example LSO in an as-worn state and including a postpartum (or pelvic) strap, a cold/hot pack, and an oval-shaped anterior panel, in accordance with an embodiment of the invention;

FIG. 41A, FIG. 41B, FIG. 41C, and FIG. 41D illustrate a perspective view, a front view, a side view, and an end view, respectively, of an example posterior panel pad of the LSO, in accordance with an embodiment of the invention;

FIG. 42 illustrates a front perspective view of an example LSO in an expanded state and including the example posterior panel pad shown in FIG. FIG. 41A, in accordance with an embodiment of the invention;

FIG. 43, FIG. 44, FIG. 45, and FIG. 46 shows an example LSO when in use, in accordance with an embodiment of the invention; and

FIG. 47 illustrates a flow diagram of an example method of using the LSO shown and described in FIG. 1 through FIG. 46, in accordance with an embodiment of the invention.

### DETAILED DESCRIPTION

The subject matter now will be described more fully hereinafter with reference to the accompanying drawings, in which some, but not all embodiments of the subject matter are shown. Like numbers refer to like elements throughout. The subject matter may be embodied in many different forms and should not be construed as limited to the embodiments set forth herein; rather, these embodiments are provided so that this disclosure will satisfy applicable legal requirements. Indeed, many modifications and other embodiments of the subject matter set forth herein will come to mind to one skilled in the art to which the subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims.

Generally, a lumbosacral orthosis (LSO) device may be any back brace or belt that supports the mid to lower spine and helps relieve back pain. For example, LSOs may be used to treat a variety of conditions, such as, but not limited to, spinal fractures, deformities, severe pain, and lumbar plexus injuries. LSOs may be used to support spinal posture, help reduce pain, prevent further injury, and promote healing.

However, in some embodiments, the subject matter here provides an LSO and method that, in one example, may be used by women both during pregnancy and/or after pregnancy (i.e., postpartum).

In some embodiments, the LSO and method provide a compression garment (brace/belt) designed to be used both during pregnancy and after pregnancy (i.e., postpartum).

In some embodiments, the LSO and method provide a compression garment that (1) may be used by women to relieve pregnancy-induced pressure from the abdomen, hips, and lower back to reinforce the body's core and thereby providing comfort and relief throughout pregnancy, and (2) may be used by women for postpartum support that provides postpartum pain relief.

In some embodiments, the LSO and method provide a compression garment that may be used to relieve pain associated with natural changes that may occur during pregnancy; natural changes such as increased spinal curvature because of anterior pelvic tilt and increased lordotic curve.

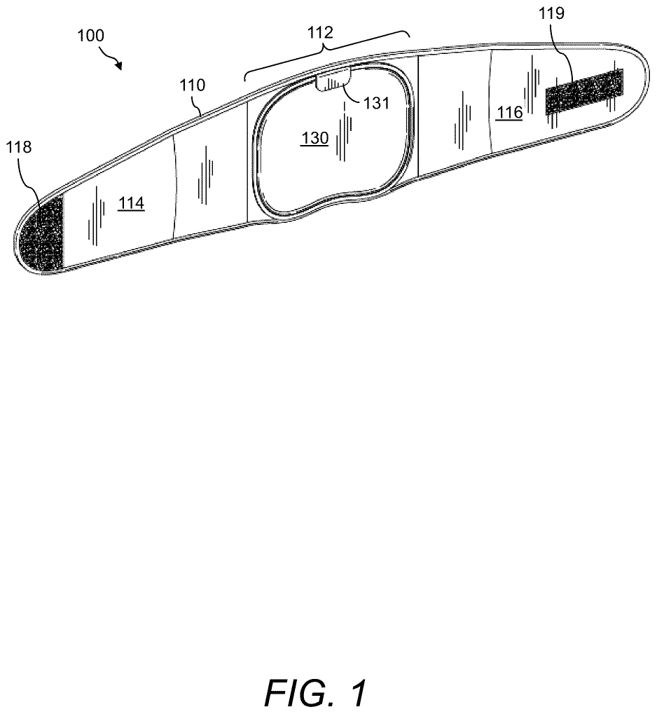

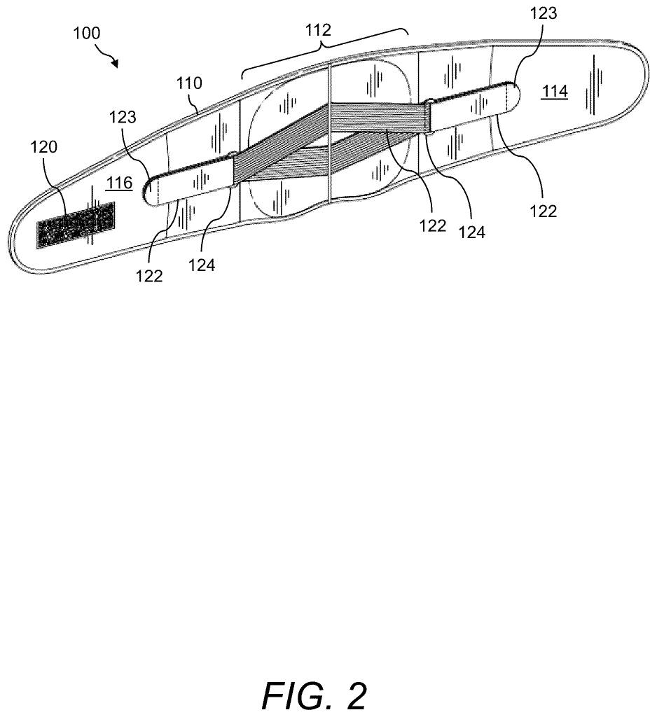

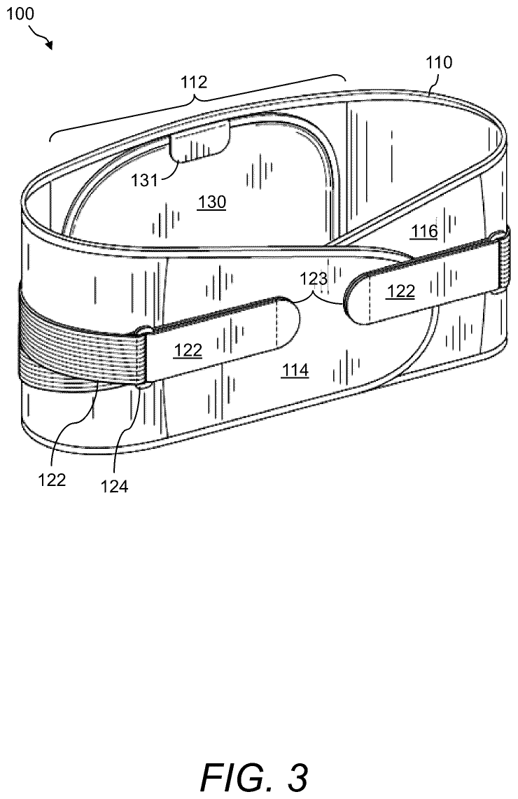

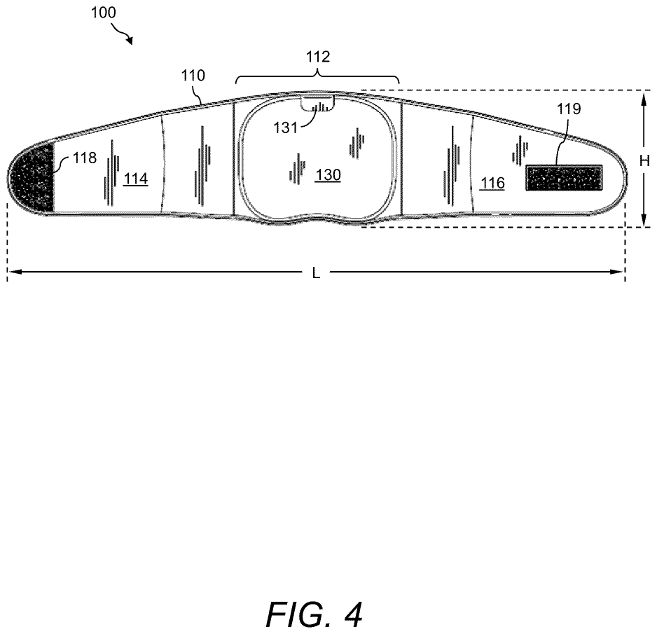

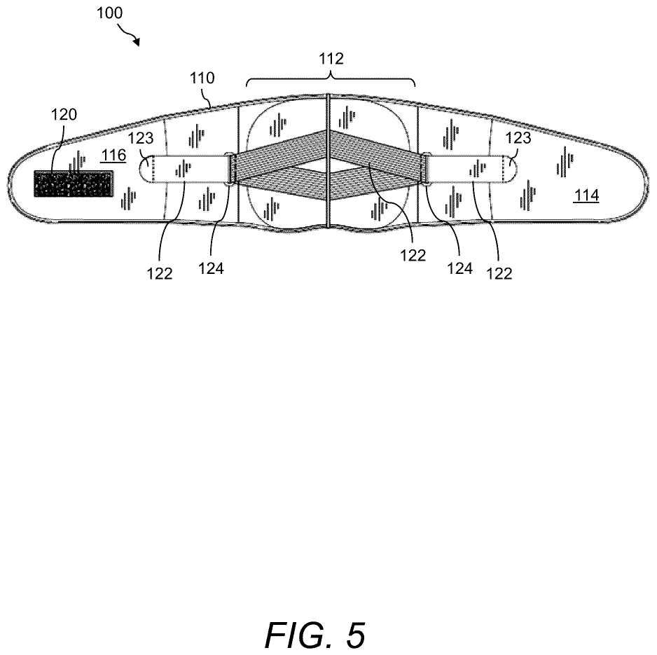

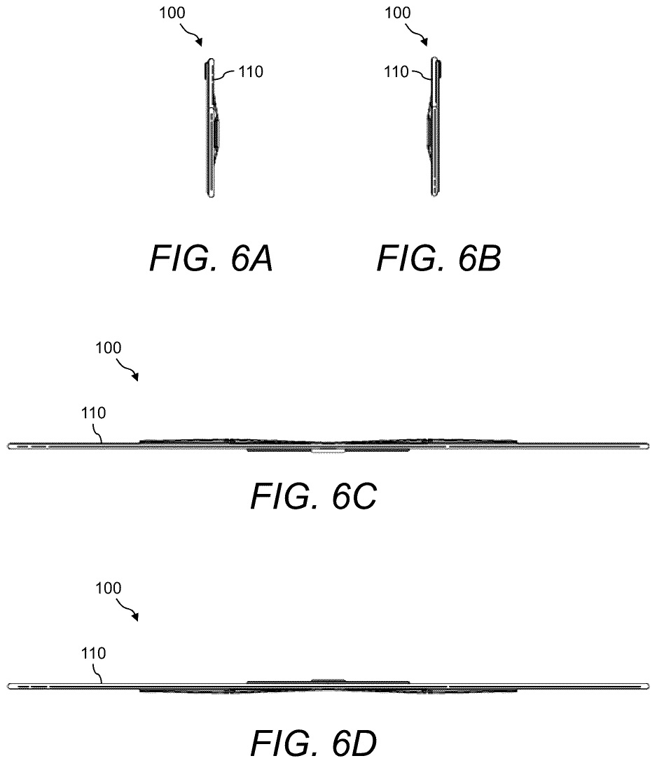

Referring now to FIG. 1 through FIG. 6D is various views of an example of a lumbosacral orthosis (LSO) 100, in accordance with an embodiment of the invention. For example, FIG. 1 and FIG. 2 show a front perspective view and a back perspective view, respectively, of the LSO 100 in an expanded state. FIG. 3 shows a perspective view of the LSO 100 in an as-worn state. FIG. 4 and FIG. 5 show a front plan view and a back plan view, respectively, of the LSO 100 an expanded state. FIG. 6A, FIG. 6B, FIG. 6C, and FIG. 6D show a first end view, a second end view, a first side view, and a second side view, respectively, of the LSO 100 in an expanded state.

In the example shown in FIG. 1 through FIG. 6D, the LSO 100 may include an orthotic support band 110 formed by a central portion 112 flanked by a right portion 114 and a left portion 116.

Further, FIG. 1 shows a connection mechanism 118, such as a hook and loop patch, may be provided on the inside surface of the right portion 114 of the orthotic support band 110. The connection mechanism 118 may be used to secure the right portion 114 of the orthotic support band 110 to the outside surface of the left portion 116 when worn by the user (see FIG. 3). Alternatively, the connection mechanism 118, may be provided on the inside surface of the left portion 116 of the orthotic support band 110, and the connection mechanism 118 may be used to secure the left portion 116 of the orthotic support band 110 to the outside surface of the right portion 114. Here and subsequently, any references to “hook and loop” may mean, for example, Velcro® material.

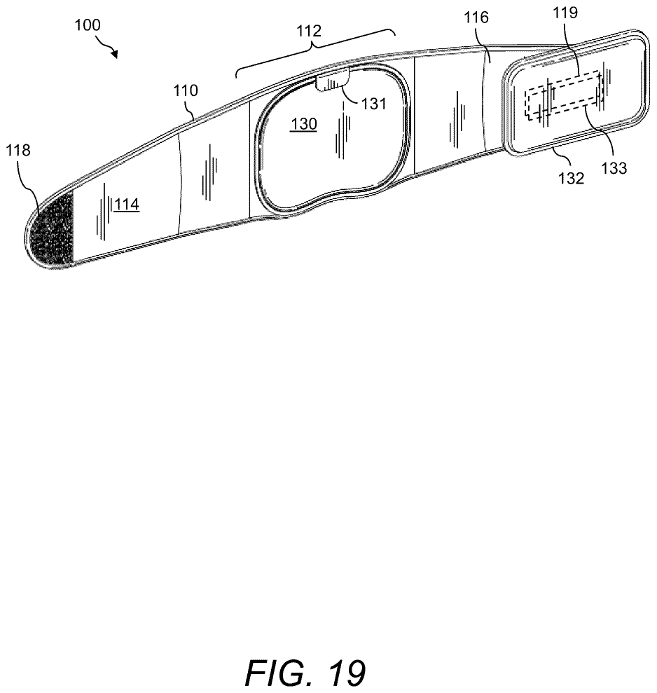

Further, FIG. 1 shows a connection mechanism 119, such as a hook and loop patch, may be provided on the inside (and/or outside) surface of the left portion 116 of the orthotic support band 110. The attachment mechanism 119 may be used to secure an optional anterior (or front or abdominal) panel 132, as shown, for example, in FIG. 19 through FIG. 28D. Accordingly, the anterior (or front or abdominal) panel 132 may include an attachment mechanism 133, such as a hook and loop patch, for connecting to the attachment mechanism 119 of the orthotic support band 110. Examples of anterior (or front or abdominal) panels 132 are shown below in FIG. 19 through FIG. 23D and FIG. 24 through FIG. 28D. Alternatively, connection mechanism 119 may be provided on the inside (and/or outside) surface of the right portion 114 of the orthotic support band 110.

Further, FIG. 2 shows an attachment mechanism 120, such as a hook and loop patch, may be provided on the outside surface of the left portion 116 of the orthotic support band 110. The attachment mechanism 120 may be used to secure the left portion 116 of the orthotic support band 110 to the inside surface of the right portion 114 when worn by the user (see FIG. 3). Alternatively, attachment mechanism 120 may be provided on the outside surface of the right portion 114 of the orthotic support band 110. The attachment mechanism 120 may be used to secure the right portion 114 of the orthotic support band 110 to the inside surface of the left portion 116.

Further, FIG. 2 shows the LSO 100 may include an adjustable pull strap 122 that wraps around the back (or outside) of the orthotic support band 110. The adjustable pull strap 122 may be formed, for example, of nylon webbing material. Further, the adjustable pull strap 122 may be adjustable via a pair of sliding buckles 124. Further, each inside end of the adjustable pull strap 122 may include an attachment mechanism 123, such as a hook and loop patch. The attachment mechanisms 123 of the adjustable pull strap 122 may be used to connect to any outside surface of the orthotic support band 110 when worn (see FIG. 3).

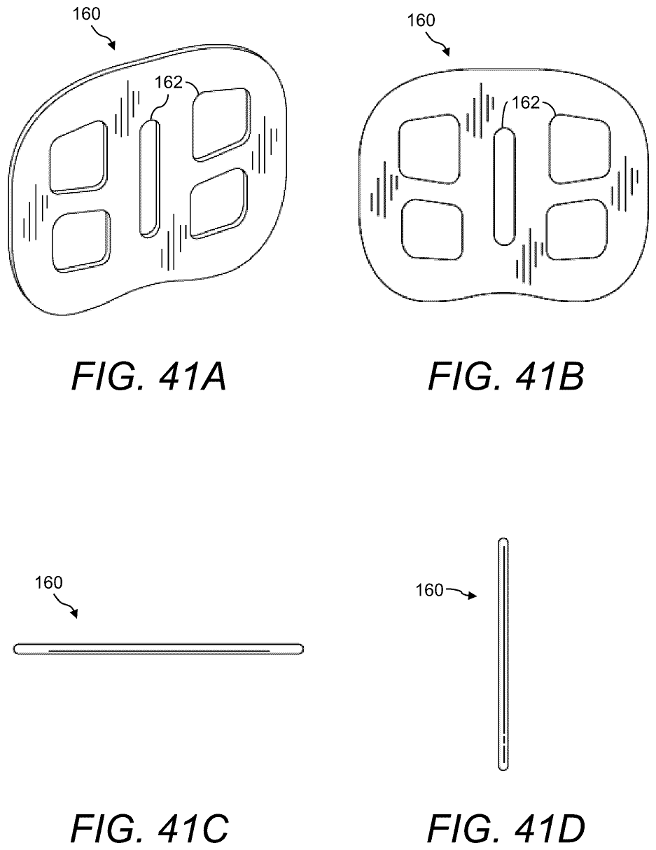

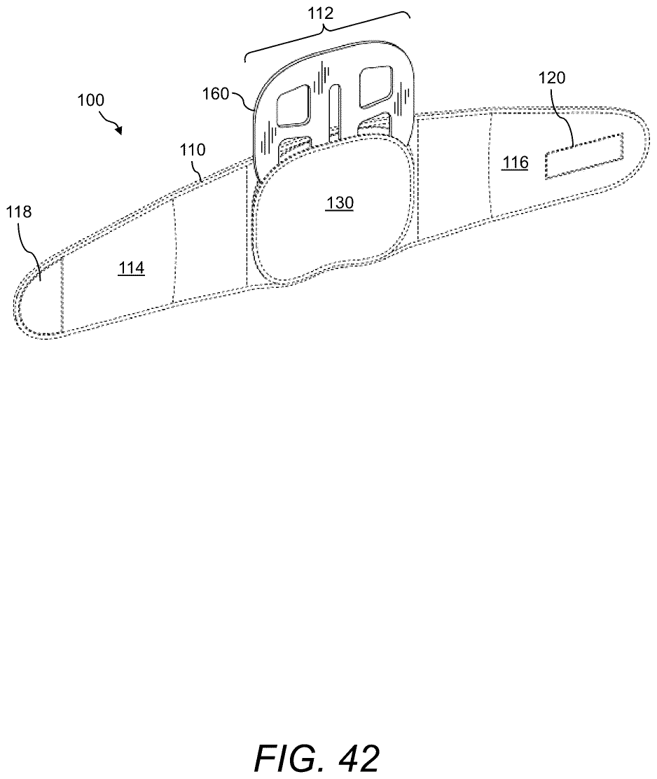

Additionally, the LSO 100 may include a posterior (or back) panel pouch 130 at the inside of the central portion 112 of the orthotic support band 110. The posterior (or back) panel pouch 130 may include a closing mechanism 131, such as a hook and loop patch. The posterior (or back) panel pouch 130 may be provided for holding an optional posterior panel pad 160, as shown, for example, in FIG. 42. The posterior panel pad 160 may be permanently (or removably) installed within the posterior (or back) panel pouch 130. An example of the posterior panel pad 160 is shown below in FIG. 41A through FIG. 42.

Referring to FIG. 4, the orthotic support band 110 of the LSO 100 may have an overall length L and an overall height H. Accordingly, the LSO 100 may be provided in multiple sizes to support all users. Non-limiting example estimated sizes are shown below in Table 1.

TABLE 1
Example sizes of the LSO 100 (estimated)

| | Measurement Around | |
|---|---|---|
| Size | Belly | |
| Small | L = 26-33 inches | H = 9.5-10.5 inches (24-27 cm) |
| (S) | (66-84 cm) | |
| Medium | L = 32-40 inches | H = 9.5-10.5 inches (24-27 cm) |
| (M) | (81-102 cm) | |
| Large | L = 39-48 inches | H = 9.5-10.5 inches (24-27 cm) |
| (L) | (99-122 cm) | |
| Extra-large | L = 47-56 inches | H = 9.5-10.5 inches (24-27 cm) |
| (XL) | (119-142 cm) | |

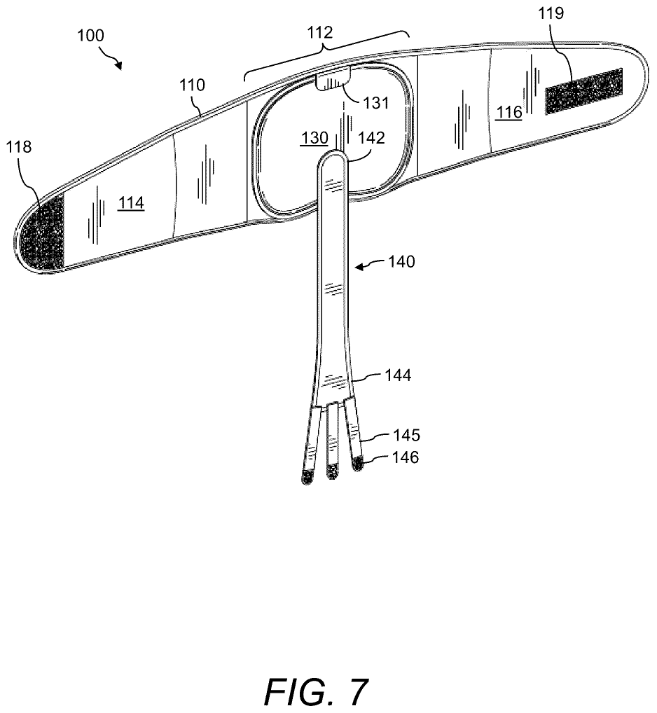

The LSO 100 shown and described above in FIG. 1 through FIG. 6D may include various other components that may be used optionally and in various configurations. Examples of which are shown below in FIG. 7 through FIG. 41D.

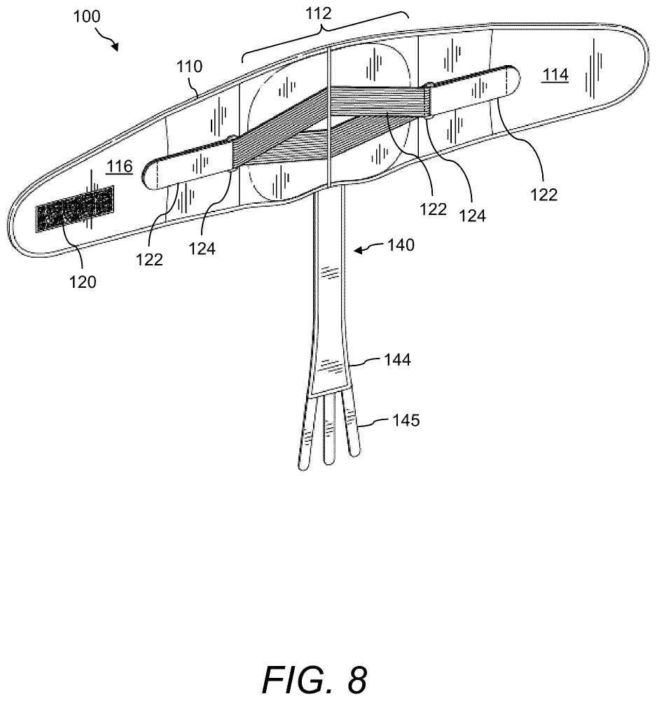

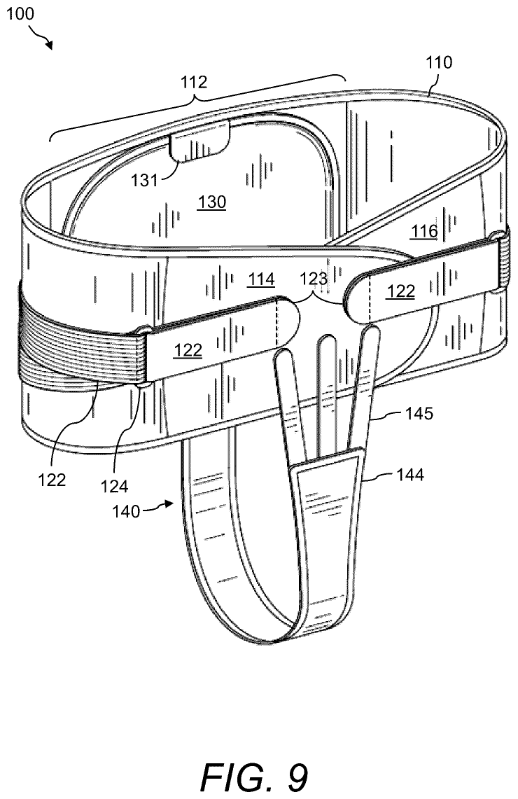

Referring now to FIG. 7, FIG. 8, and FIG. 9 is an example configuration of the LSO 100 that further includes a postpartum (or pelvic) strap 140. For example, FIG. 7 and FIG. 8 show a front perspective view and a back perspective view, respectively, of the LSO 100 in an expanded state and including the postpartum (or pelvic) strap 140. FIG. 9 shows a perspective view of the LSO 100 in an as-worn state and including the postpartum (or pelvic) strap 140.

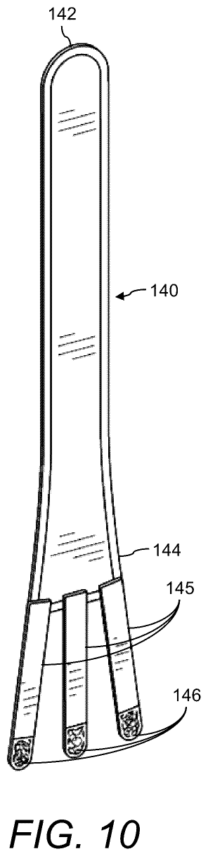

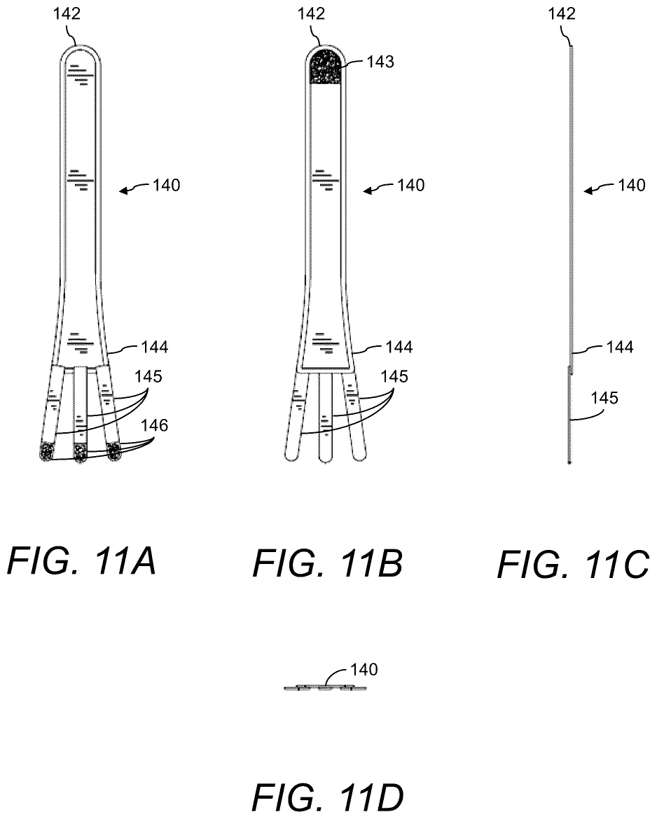

Further, FIG. 10 shows a perspective view of an example of the postpartum (or pelvic) strap 140. Additionally, FIG. 11A, FIG. 11B, FIG. 11C, and FIG. 11D show a front plan view, a back plan view, a side view, and an end view, respectively, of the postpartum (or pelvic) strap 140 shown in FIG. 10. In this example, the postpartum (or pelvic) strap 140 may include a posterior end 142 and an anterior end 144. The anterior end 144 of postpartum (or pelvic) strap 140 may include one or more (e.g., three) finger straps 145. Further, the posterior end 142 of the postpartum (or pelvic) strap 140 may include an attachment mechanism 143, such as a hook and loop patch (see FIG. 11B). Similarly, each of the one or more finger straps 145 may include an attachment mechanism 146, such as a hook and loop patch (see FIG. 11A).

Referring again to FIG. 7, FIG. 8, and FIG. 9, the posterior end 142 of the postpartum (or pelvic) strap 140 may be attached to the inside of the posterior (or back) panel pouch 130. Then, when the LSO 100 is worn (see FIG. 9), the postpartum (or pelvic) strap 140 may be stretched from back to front between the user's legs (not shown) and the one or more finger straps 145 attached to the outside (or inside) of the right portion 114 of the orthotic support band 110 via the attachment mechanisms 146. The one or more finger straps 145 may alternatively be attached to the outside of the left portion 116 of the orthotic support band 110 via the attachment mechanisms 146.

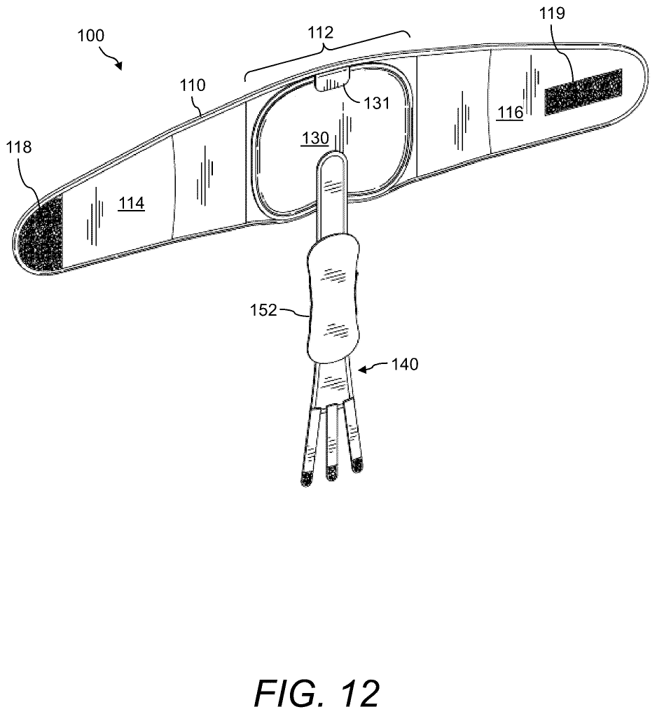

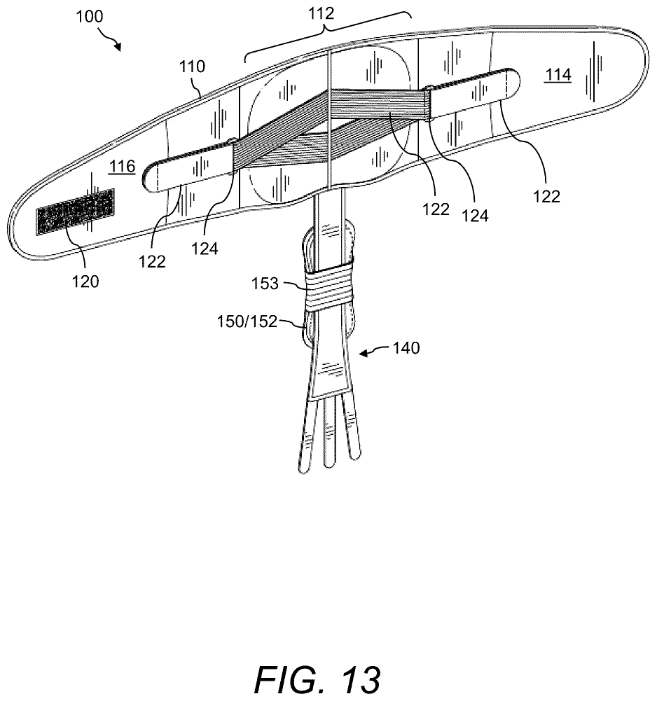

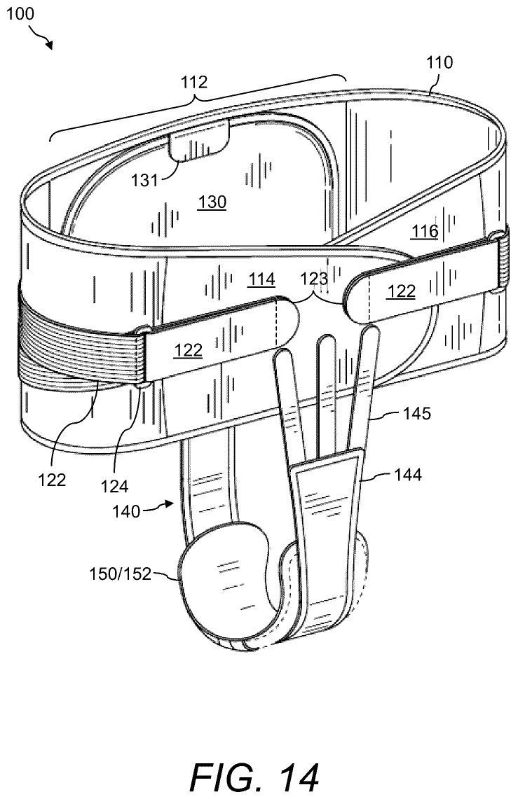

Referring now to FIG. 12, FIG. 13, and FIG. 14 is another example configuration of the LSO 100 that includes the postpartum (or pelvic) strap 140 with a cold/hot pack. For example, FIG. 12 and FIG. 13 shows a front perspective view and a back perspective view, respectively, of an example of the LSO 100 in an expanded state and including both the postpartum (or pelvic) strap 140 and a cold/hot pack 150 held in a cold/hot pack pocket 152. FIG. 14 shows a perspective view of an example of the LSO 100 in an as-worn state and including both the postpartum (or pelvic) strap 140 and the cold/hot pack 150 held in the cold/hot pack pocket 152.

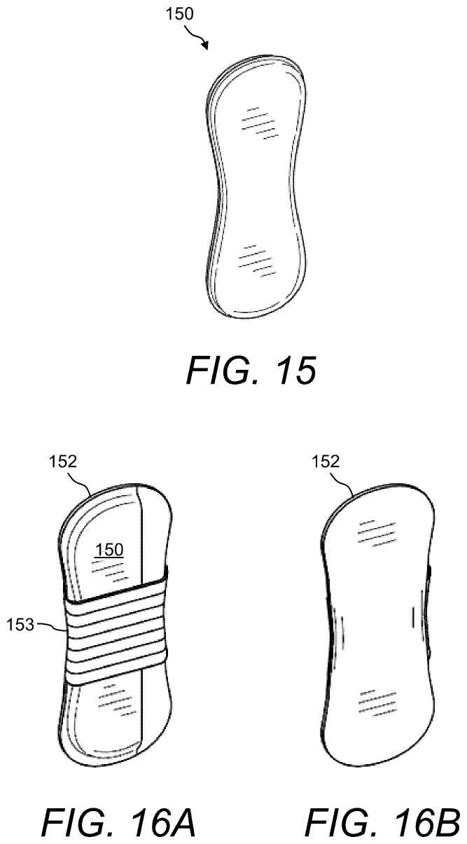

Further, FIG. 15 shows a perspective view of an example of the cold/hot pack 150 of the LSO 100. FIG. 16A and FIG. 16B show a front perspective view and a back perspective view, respectively, of an example of the cold/hot pack pocket 152. The cold/hot pack pocket 152 is designed to hold the cold/hot pack 150 and then attach to the postpartum (or pelvic) strap 140. For example, the cold/hot pack pocket 152 may include an elastic band (or loop) 153 that can be installed around the postpartum (or pelvic) strap 140 for holding the cold/hot pack pocket 152 and the cold/hot pack 150 in place (see FIG. 14). In this way, the cold/hot pack 150 may be positioned as desired anywhere along the postpartum (or pelvic) strap 140.

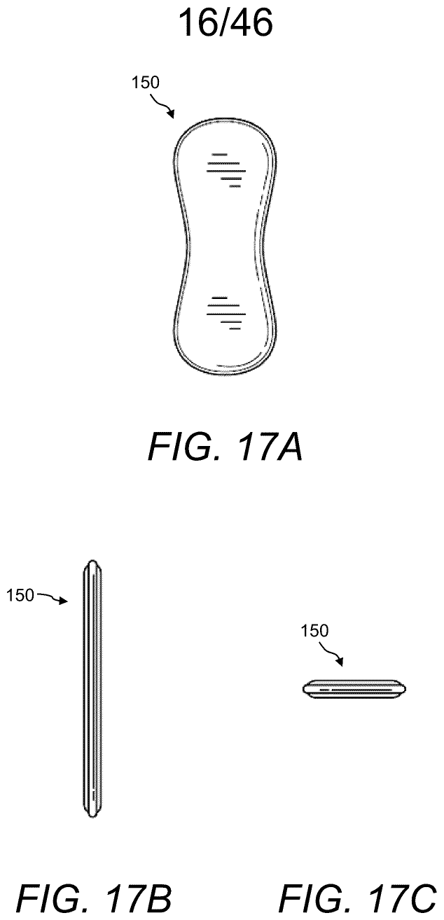

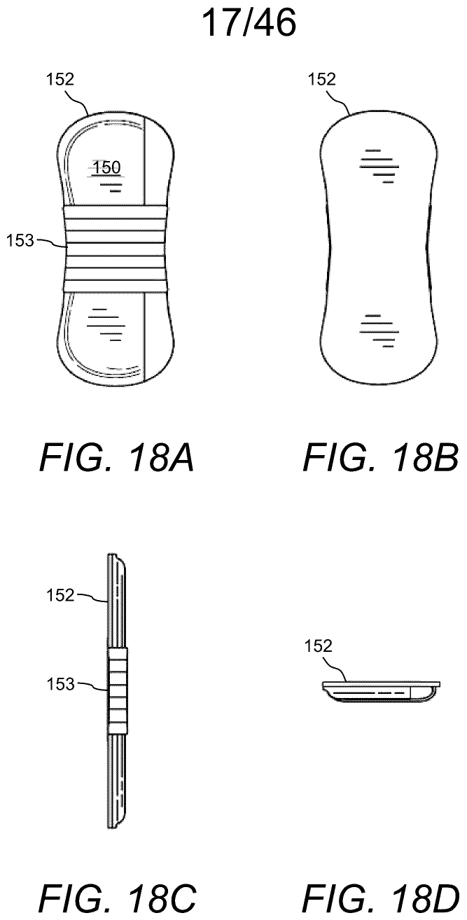

Further to the example, FIG. 17A, FIG. 17B, and FIG. 17C show a front view, a side view, and an end view, respectively, of the cold/hot pack 150 shown in FIG. 15. Additionally, FIG. 18A, FIG. 18B, FIG. 18C, and FIG. 18D show a front view, a back view, a side view, and an end view, respectively, of the cold/hot pack pocket 152 shown in FIG. 16A and FIG. 16B.

Referring still to FIG. 12 through FIG. 18D, the postpartum (or pelvic) strap 140 with the cold/hot pack 150 may be useful to provide additional pelvic support, ease perineal and muscle soreness, and reduce inflammation and pain.

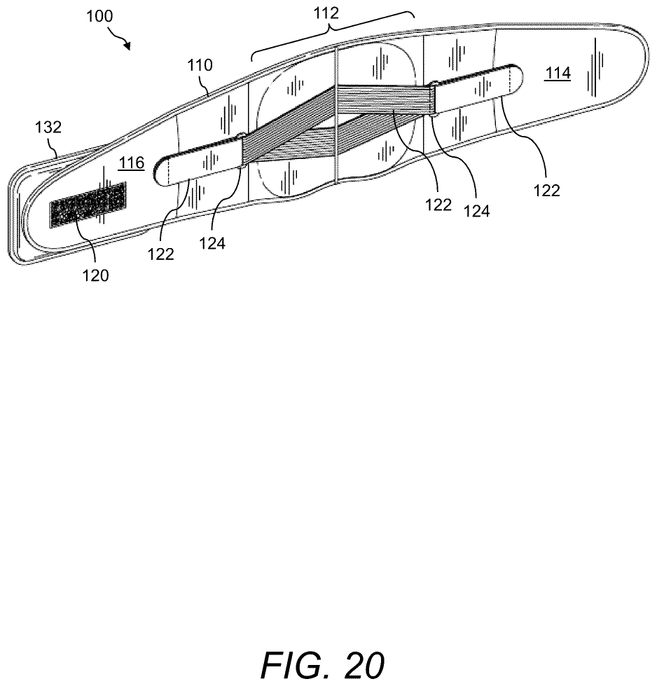

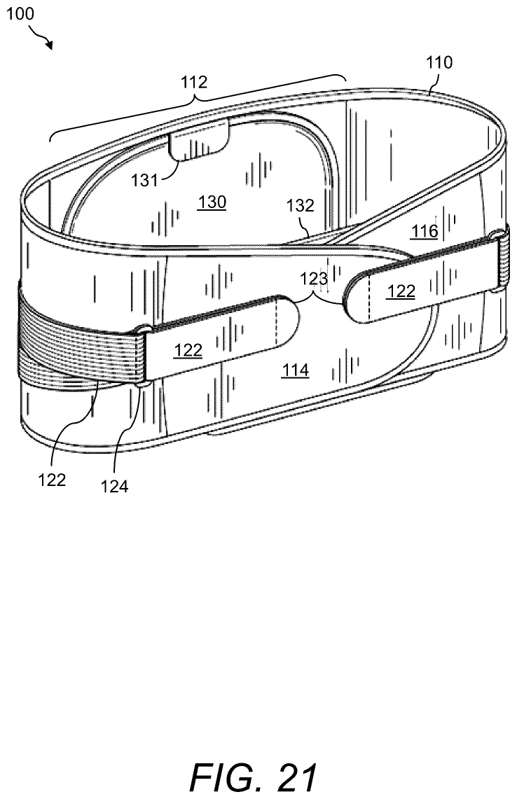

Referring now to FIG. 19, FIG. 20, and FIG. 21 is yet another example configuration of the LSO 100 that includes a rectangle-shaped anterior panel 132. For example, FIG. 19 and FIG. 20 show a front perspective view and a back perspective view, respectively, of the LSO 100 in an expanded state and including an example of the rectangle-shaped anterior panel 132. FIG. 21 shows a perspective view of the LSO 100 in an as-worn state and including the rectangle-shaped anterior panel 132.

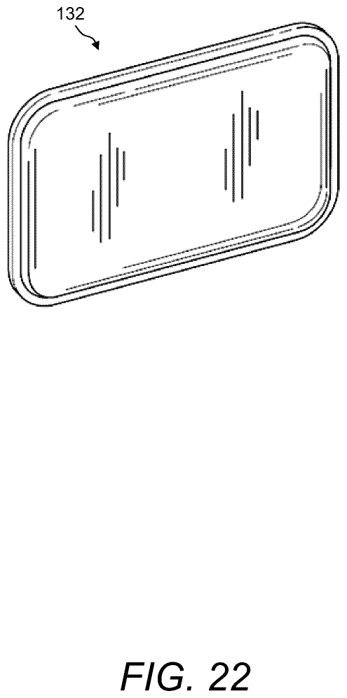

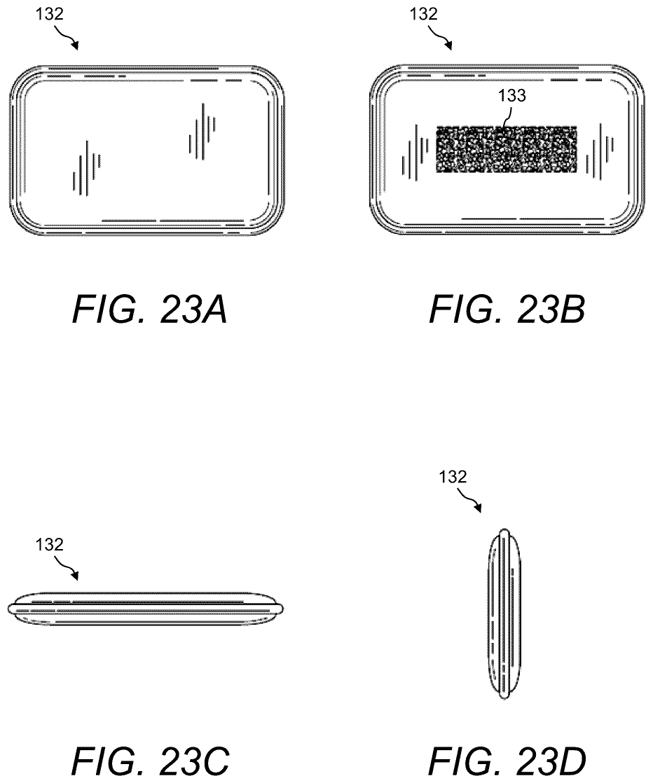

Further, FIG. 22 shows a perspective view of an example of the rectangle-shaped anterior panel 132. FIG. 23A, FIG. 23B, FIG. 23C, and FIG. 23D show a front view, a back view, a side view, and an end view, respectively, of the rectangle-shaped anterior panel 132 shown in FIG. 22.

The rectangle-shaped anterior panel 132 may be a substantially rectangular padded panel of any size. That is, the rectangle-shaped anterior panel 132 may be provided in different sizes. FIG. 19 and FIG. 23B show that the rectangle-shaped anterior panel 132 may include an attachment mechanism 133, such as a hook and loop patch. The attachment mechanism 133 may be used to connect the rectangle-shaped anterior panel 132 to the attachment mechanism 119 at the inside of the orthotic support band 110, as shown in FIG. 19.

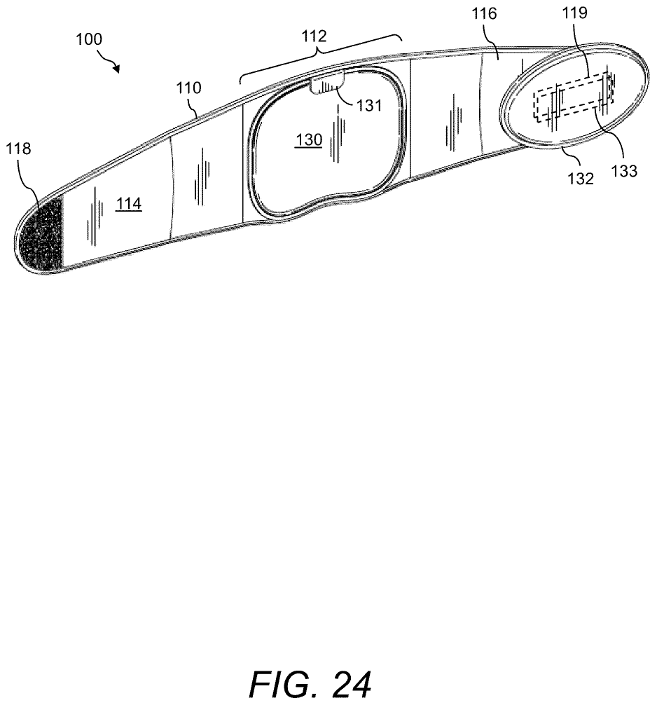

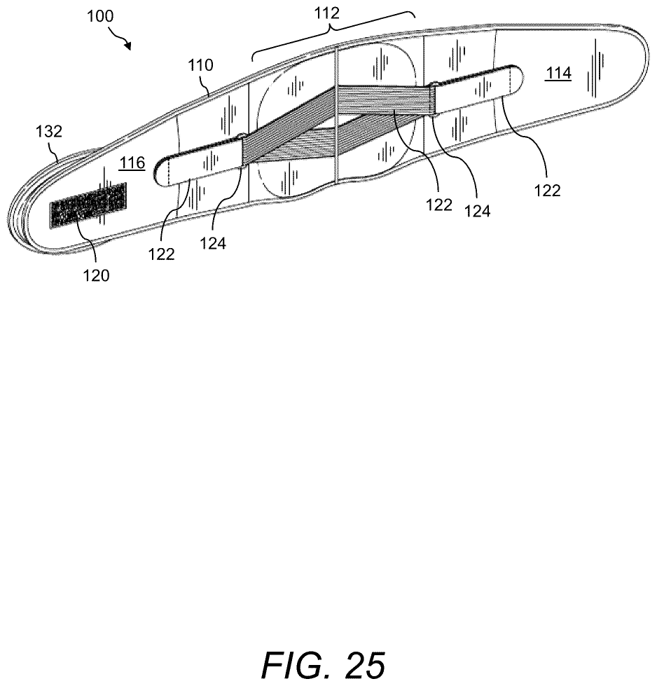

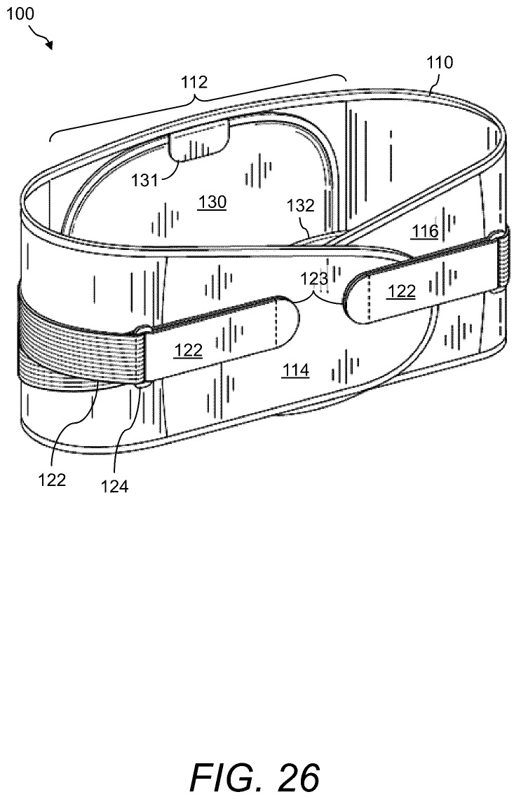

The anterior panel 132 of the LSO 100 is not limited to rectangle-shaped. The anterior panel 132 may be any shape. Referring now to FIG. 24, FIG. 25, and FIG. 26 is yet another example configuration of the LSO 100 that includes an oval-shaped anterior panel 132. For example, FIG. 24 and FIG. 25 show a front perspective view and a back perspective view, respectively, of the LSO 100 in an expanded state and including an example of the oval-shaped anterior panel 132. FIG. 26 shows a perspective view of the LSO 100 in an as-worn state and including the oval-shaped anterior panel 132.

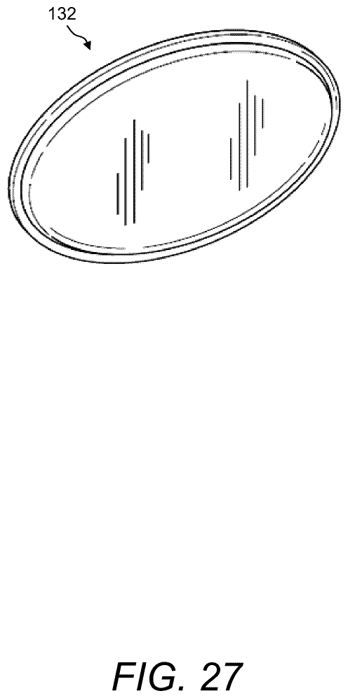

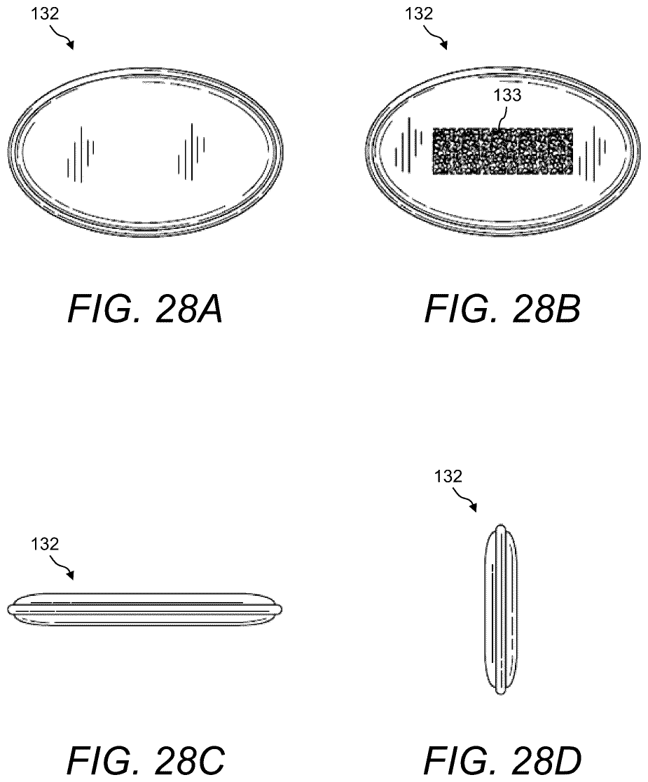

Further, FIG. 27 shows a perspective view of an example of the oval-shaped anterior panel 132. FIG. 28A, FIG. 28B, FIG. 28C, and FIG. 28D show a front view, a back view, a side view, and an end view, respectively, of the oval-shaped anterior panel 132 shown in FIG. 27.

The oval-shaped anterior panel 132 may be a substantially oval padded panel of any size. That is, the oval-shaped anterior panel 132 may be provided in different sizes. FIG. 24 and FIG. 28B show that the oval-shaped anterior panel 132 may include an attachment mechanism 133, such as a hook and loop patch. The attachment mechanism 133 may be used to connect the oval-shaped anterior panel 132 to the attachment mechanism 119 at the inside of the orthotic support band 110, as shown in FIG. 24.

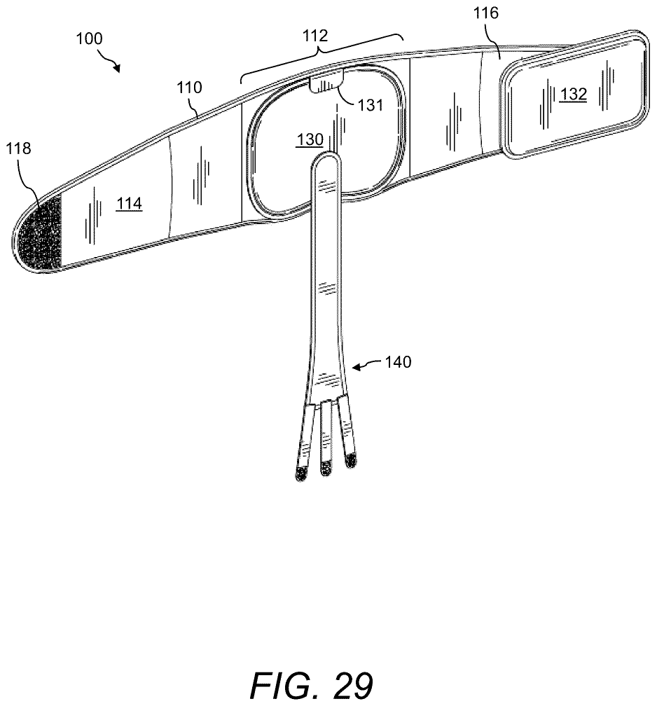

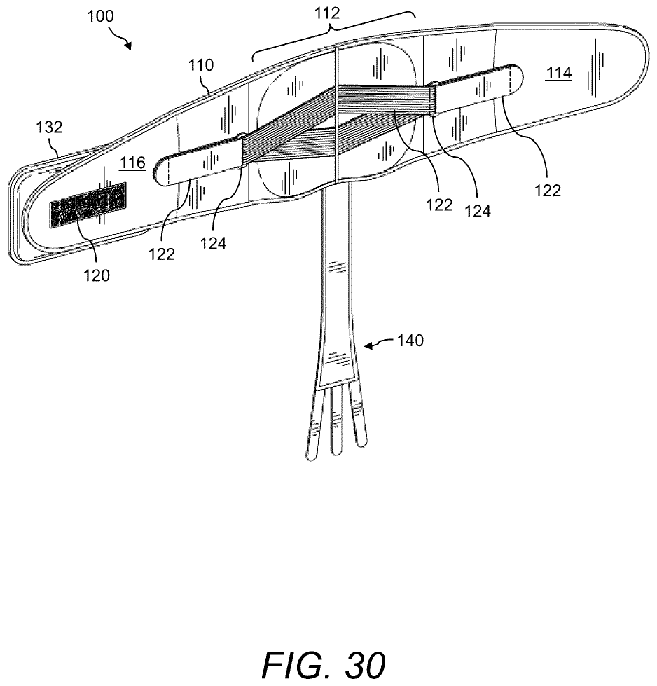

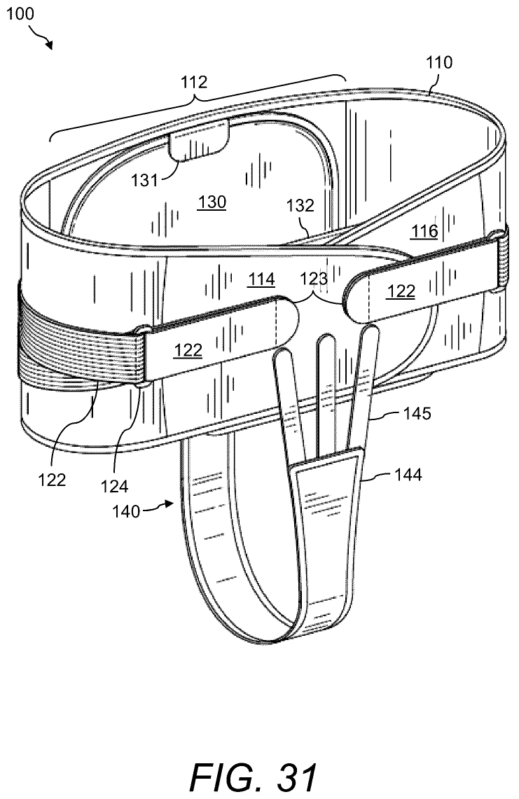

Referring now to FIG. 29, FIG. 30, and FIG. 31 is yet another example configuration of the LSO 100 that includes both the postpartum (or pelvic) strap 140 and the rectangle-shaped anterior panel 132. For example, FIG. 29 and FIG. 30 show a front perspective view and a back perspective view, respectively, of the LSO 100 in an expanded state and including both the postpartum (or pelvic) strap 140 and the rectangle-shaped anterior panel 132. FIG. 31 shows a perspective view of an example of the LSO 100 in an as-worn state and including both the postpartum (or pelvic) strap 140 and the rectangle-shaped anterior panel 132.

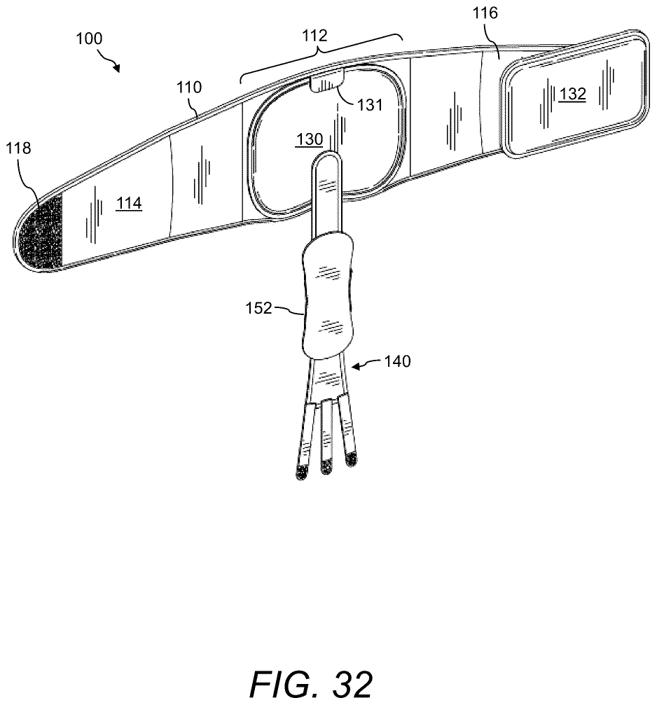

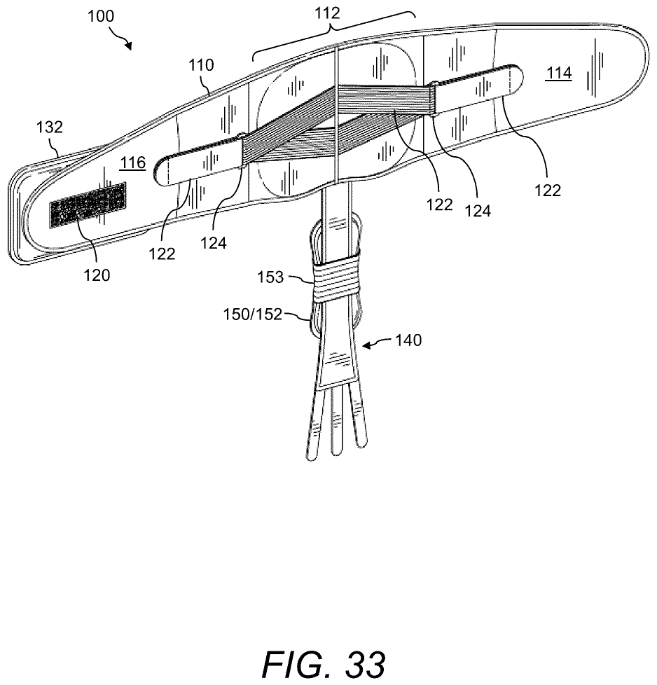

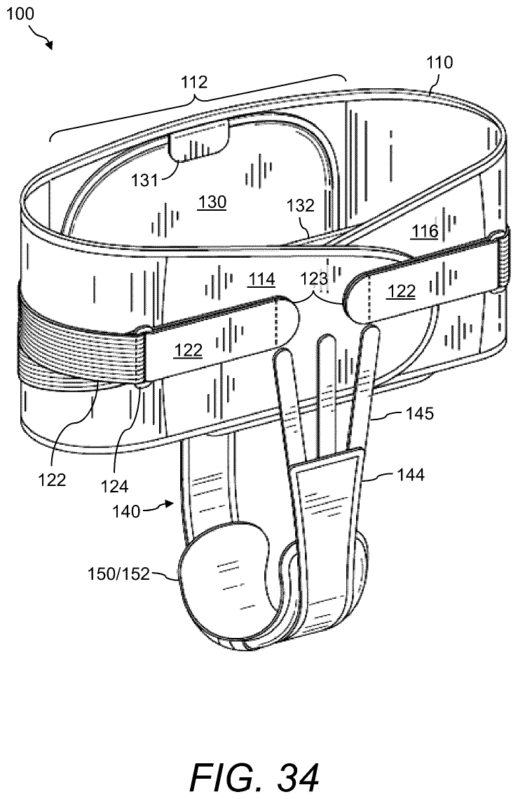

Referring now to FIG. 32, FIG. 33, and FIG. 34 is yet another example configuration of the LSO 100 that includes the postpartum (or pelvic) strap 140, the cold/hot pack 150, and the rectangle-shaped anterior panel 132. For example, FIG. 32 and FIG. 33 show a front perspective view and a back perspective view, respectively, of the LSO 100 in an expanded state and including the postpartum (or pelvic) strap 140, the cold/hot pack 150, and the rectangle-shaped anterior panel 132. FIG. 34 shows a perspective view of the LSO 100 in an as-worn state and including the postpartum (or pelvic) strap 140, the cold/hot pack 150, and the rectangle-shaped anterior panel 132.

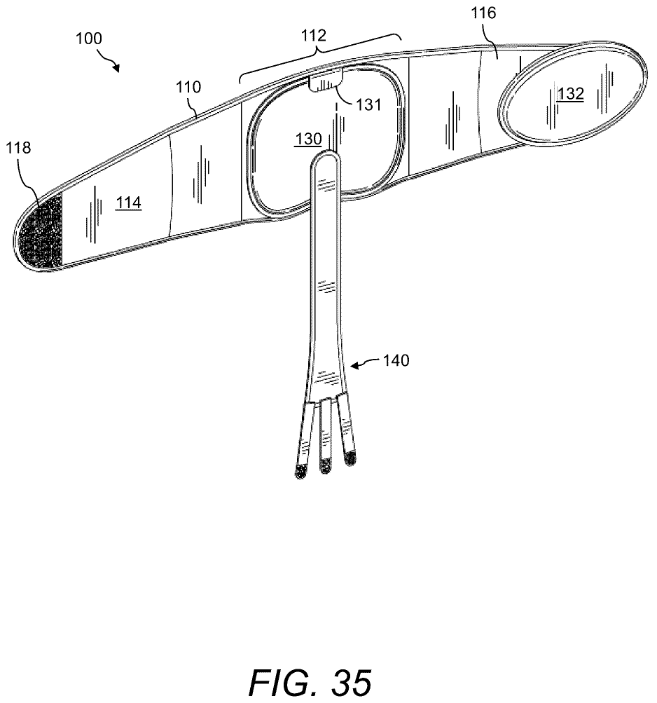

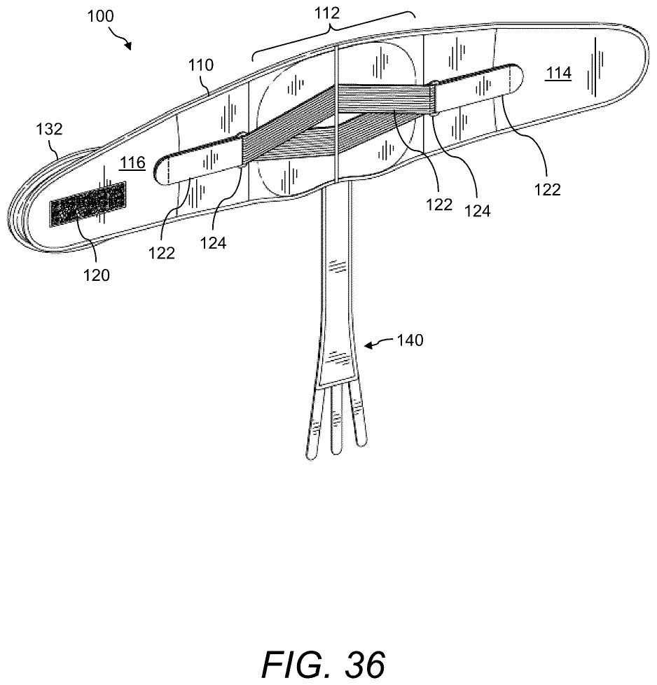

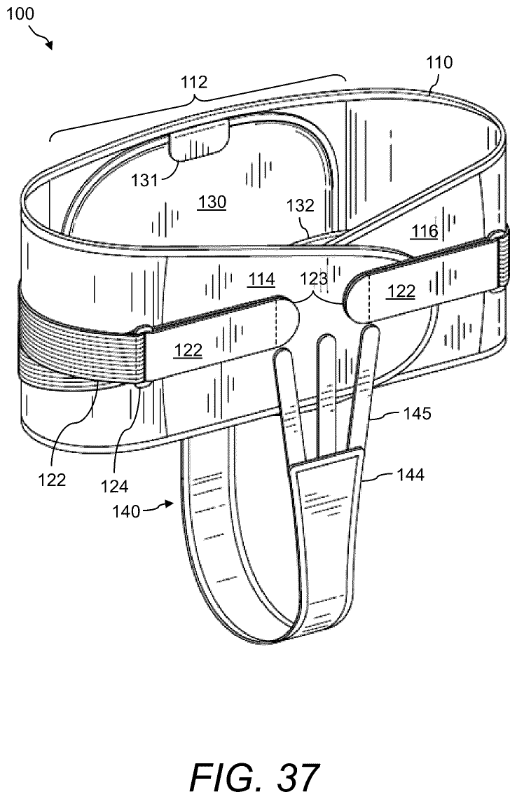

Referring now to FIG. 35, FIG. 36, and FIG. 37 is yet another example configuration of the LSO 100 that includes both the postpartum (or pelvic) strap 140 and the oval-shaped anterior panel 132. For example, FIG. 35 and FIG. 36 show a front perspective view and a back perspective view, respectively, of the LSO 100 in an expanded state and including both the postpartum (or pelvic) strap 140 and the oval-shaped anterior panel 132. FIG. 37 shows a perspective view of the LSO 100 in an as-worn state and including both the postpartum (or pelvic) strap 140 and the oval-shaped anterior panel 132.

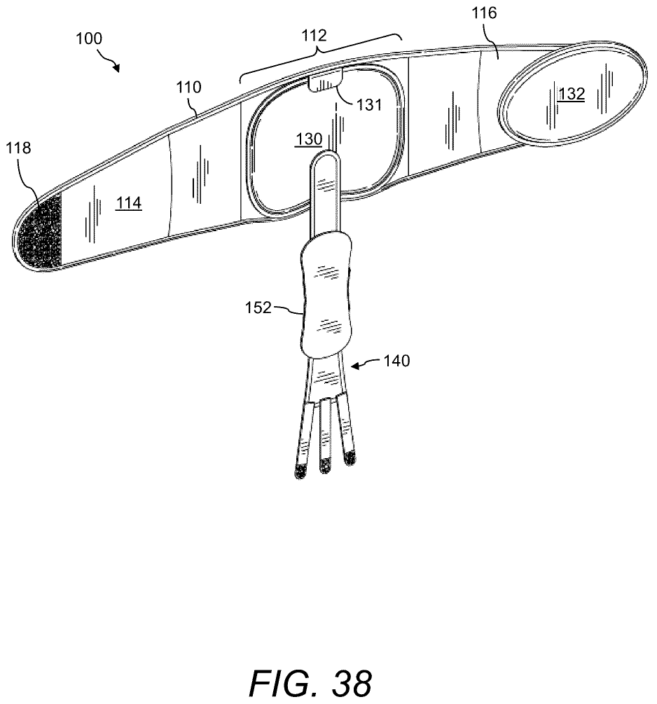

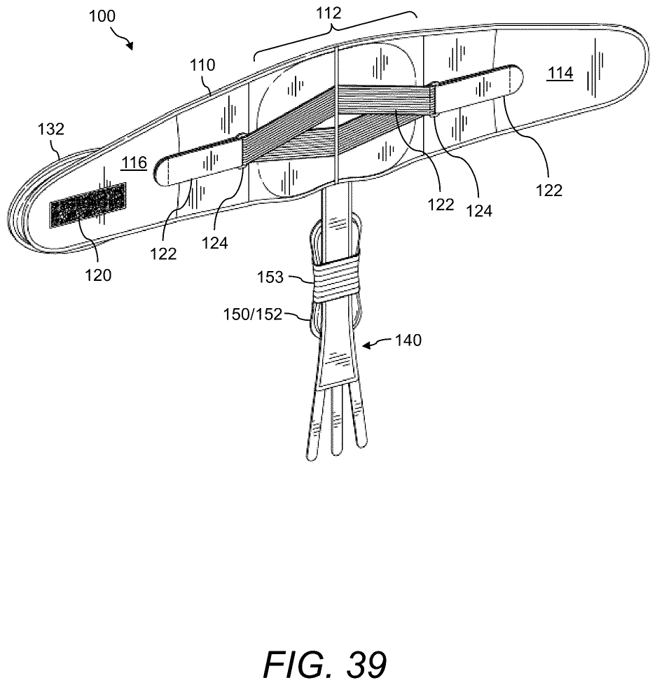

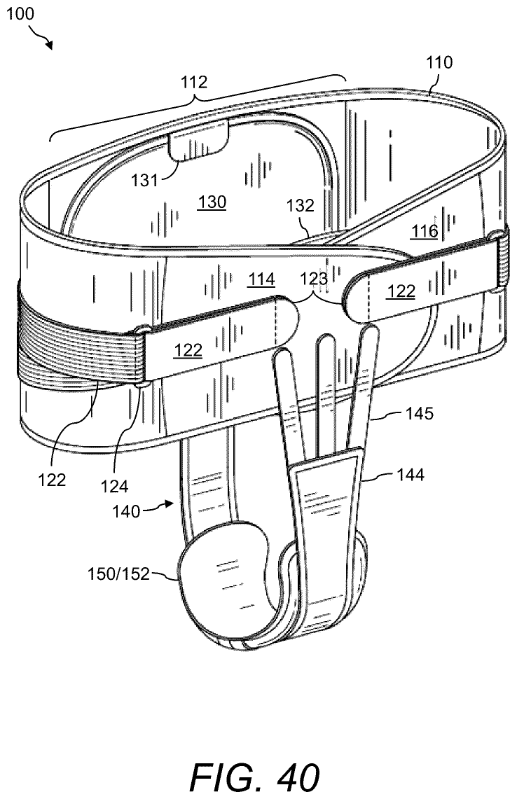

Referring now to FIG. 38, FIG. 39, and FIG. 40 is still another example configuration of the LSO 100 that includes the postpartum (or pelvic) strap 140, the cold/hot pack 150, and the oval-shaped anterior panel 132. For example, FIG. 38 and FIG. 39 show a front perspective view and a back perspective view, respectively, of the LSO 100 in an expanded state and including the postpartum (or pelvic) strap 140, the cold/hot pack 150, and the oval-shaped anterior panel 132. FIG. 40 illustrates a perspective view of the LSO 100 in an as-worn state and including the postpartum (or pelvic) strap 140, the cold/hot pack 150, and the oval-shaped anterior panel 132.

Referring now to FIG. 41A, FIG. 41B, FIG. 41C, and FIG. 41D is a perspective view, a front view, a side view, and an end view, respectively, of an example of a posterior panel pad 160 of the LSO 100, in accordance with an embodiment of the invention.

The posterior panel pad 160 may be a solid piece or may include any arrangement of openings 162. Posterior panel pad 160 may be designed to be fitted into the posterior (or back) panel pouch 130 at the inside of the central portion 112 of the orthotic support band 110, as shown, for example, in FIG. 42. Again, the posterior panel pad 160 may be permanently (or removably) installed within the posterior (or back) panel pouch 130.

In one example, the posterior panel pad 160 may be a moldable posterior panel that can be reformed for a custom fit by a qualified professional. In one non-limiting example, posterior panel 130 may be made of acrylonitrile butadiene styrene (ABS) plastic.

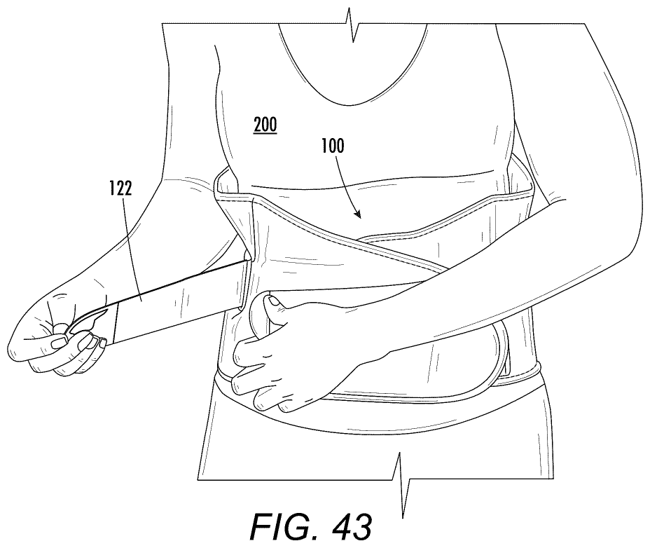

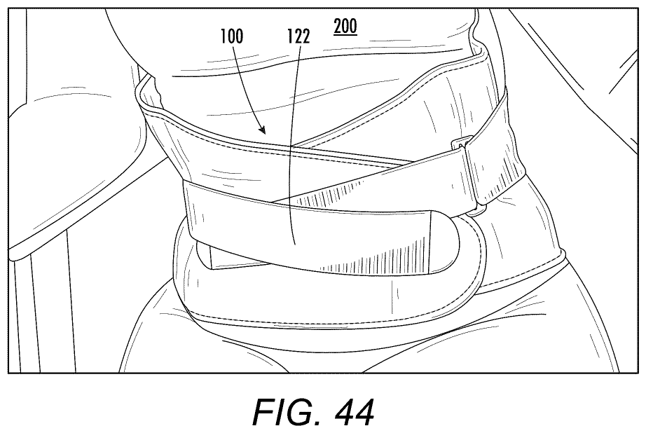

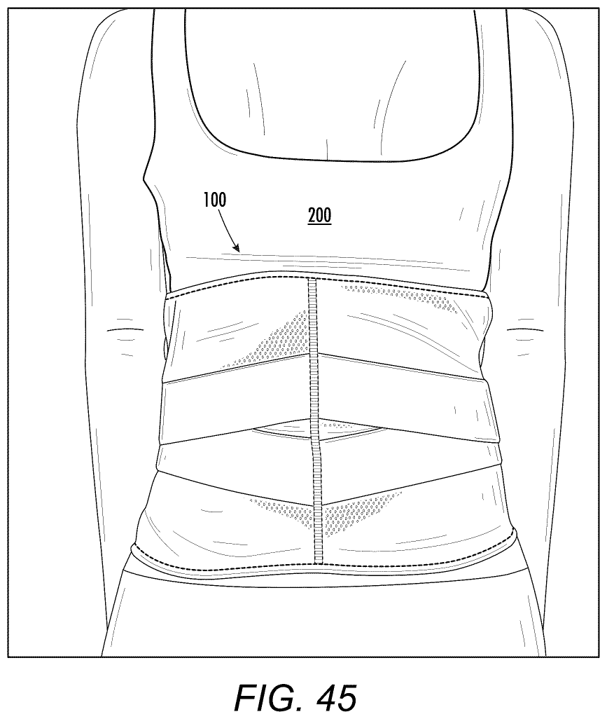

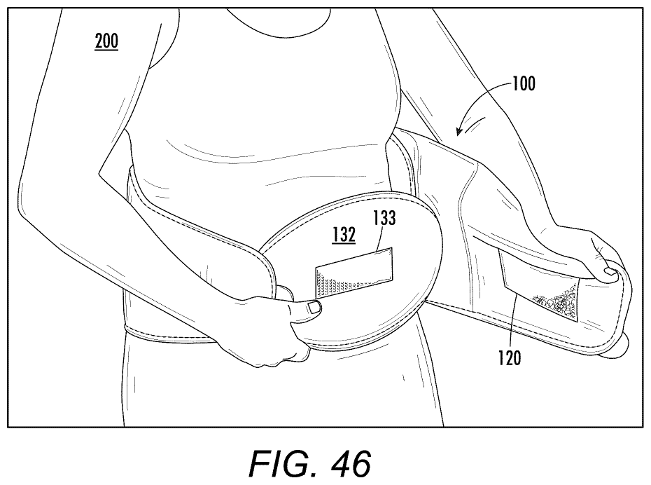

Referring now to FIG. 43, FIG. 44, FIG. 45, and FIG. 46 is photos of an example of the LSO 100 when in use, in accordance with an embodiment of the invention. The LSO 100, as shown in FIG. 43, FIG. 44, FIG. 45, and FIG. 46, is one example implementation of the LSO 100 shown in FIG. 1 through FIG. 42 that includes a full-length adjustable pull strap 122. Each end of the full-length adjustable pull strap 122 may include the attachment mechanism 123, such as a hook and loop patch, to form a full-length adjustable pull strap 122. Here, a user 200 is shown using the full-length adjustable pull strap 122 to secure the LSO 100 around her waist.

Referring now again to FIG. 1 through FIG. 46, the components of the LSO 100 may be formed, in one non-limiting example, of soft, breathable fabric, such as 65% nylon, 35% polyester construction. However, any material suitable to provide an adjustable compression garment for forming the LSO 100 may be used.

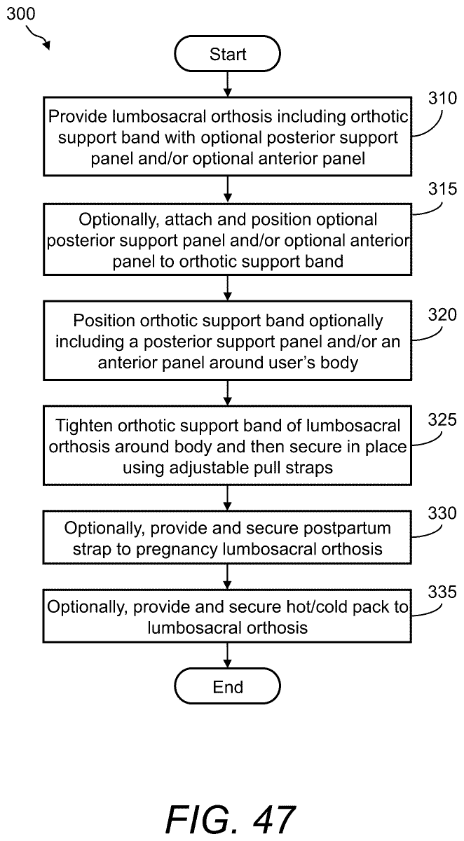

Referring now to FIG. 47 is a flow diagram of an example of a method 300 of using the LSO 100 shown and described in FIG. 1 through FIG. 46, in accordance with an embodiment of the invention. Method 300 may include, but is not limited to, the following steps.

At a step 310, a lumbosacral orthosis including an orthotic support band with an optional posterior support panel and/or anterior panel is provided. For example, the LSO 100 including the orthotic support band 110 with the optional posterior panel pad 160 may be installed in the posterior (or back) panel pouch 130 and the optional (e.g., rectangle-shaped or oval-shaped) anterior panel 132 may be provided, as described above in FIG. 1 through FIG. 46.

At a step 315, the posterior panel pad and/or anterior panel is optionally attached to the orthotic support band and positioned. In one example, the posterior panel pad 160 may be optionally installed in the posterior (or back) panel pouch 130 in a manner to be positioned at the back of a user's body, as shown, for example, in FIG. 42. In one example, the rectangle-shaped anterior panel 132 may be optionally attached to the orthotic support band 110 in a manner to be positioned at the front of a user's body, as shown, for example, in FIG. 19, FIG. 20, and FIG. 21. In another example, the oval-shaped anterior panel 132 may be optionally attached to the orthotic support band 110 in a manner to be positioned at the front of a user's body, as shown, for example, in FIG. 24, FIG. 25, and FIG. 26.

At a step 320, the orthotic support band, optionally including the posterior support panel and/or the anterior panel, is positioned around the user's body. For example, the orthotic support band 110, optionally including the posterior panel pad 160 installed in the posterior (or back) panel pouch 130 and/or the rectangle-shaped or oval-shaped anterior panel 132, may be positioned around the user's body. Examples are shown in FIG. 21, FIG. 26, FIG. 43, FIG. 44, FIG. 45, and FIG. 46.

At a step 325, the orthotic support band of the lumbosacral orthosis is tightened around the user's body and then secured in place using the adjustable pull straps. For example, the orthotic support band 110 of the LSO 100 may be tightened around the user's body and then may be secured in place using the adjustable pull strap 122. Again, examples are shown in FIG. 21, FIG. 26, FIG. 43, FIG. 44, FIG. 45, and FIG. 46.

At a step 330, optionally provide and secure a postpartum strap to the lumbosacral orthosis. For example, optionally the postpartum (or pelvic) strap 140 may be provided and then secured to the LSO 100, as shown, for example, in FIG. 9, FIG. 31, and FIG. 37. In one embodiment, the posterior end 142 of the postpartum (or pelvic) strap 140 may be attached to the inside (or outside) of the posterior (or back) panel pouch 130. Then, the postpartum (or pelvic) strap 140 may be stretched from back to front between the user's legs and the one or more finger straps 145 attached to the outside (or inside) of the right portion 114 (or left portion 116) of the orthotic support band 110.

At a step 335, optionally provide and secure a hot/cold pack to the lumbosacral orthosis. For example, optionally the cold/hot pack pocket 152 holding the cold/hot pack 150 may be provided and then secured to the postpartum (or pelvic) strap 140 of the LSO 100, as shown, for example, in FIG. 14, FIG. 34, and FIG. 40.

In summary and referring now to FIG. 1 through FIG. 47, the LSO 100 and method 300 provide a support belt or compression garment that is designed to help relieve pregnancy-induced pressure from the abdomen, hips, and lower back. The LSO 100 and method 300 may be used to reinforce the body's core, providing comfort and relief to the user throughout pregnancy and/or after pregnancy (i.e., postpartum).

Further, the orthotic support band 110 and the posterior panel pad 160 held in the posterior (or back) panel pouch 130 of the LSO 100 may be used to (1) immobilize/support the lumbosacral region of the spine, (2) reduce the load on intervertebral discs, and (3) control movement in the sagittal plane. Further, the posterior panel 130 shape may be reformed for a custom fit, for example, by a qualified professional.

Further, multiple sizes of the LSO 100 are provided to support all users. Comfortable adjustable pull straps 122 are provided to support needs throughout pregnancy and postpartum. The use of the LSO 100 helps enable the user to remain active and mobile, doing everyday activities. The adjustability of the LSO 100 provides a dynamic brace that has optional panels (e.g., the rectangle-shaped or oval-shaped anterior panel 132) to support all women throughout pregnancy and postpartum.

Further, the posterior panel pad 160 held in the posterior (or back) panel pouch 130 may be moldable and may be easily adjustable via the adjustable pull straps 122 for optimum comfort.

Further, the optional components of the LSO 100 may be provided for increased comfort and support, such as the curved anterior panel 132, the flat anterior panel 132, and/or the postpartum strap 140 that may be used to hold the cold/hot pack 150 inside the cold/hot pack pocket 152.

Further, the optional anterior panel (e.g., the rectangle-shaped or oval-shaped anterior panel 132) provides a rigid abdomen support panel. Its design helps provide additional intracavitary pressure and supports belly during pregnancy. Further, the anterior panel (e.g., the rectangle-shaped or oval-shaped anterior panel 132) may be used to disperse the baby's weight to relieve pressure on the bladder.

Further, the LSO 100 may be well-suited (1) for moms that need a higher level of support due to lumbar lordosis or back pain, (2) for moms that are expecting multiples, (3) for moms carrying large babies, and/or (4) for moms carrying low.

Further, the LSO 100 may be well-suited (1) for moms that need a higher level of support due to lumbar lordosis or back pain, (2) for moms with pre-existing spinal condition combined with back pain, (3) for moms with higher pendulous weight sometimes presenting as multiple or larger babies, (4) for moms with facet joint syndrome and or arthritis, (5) for moms with hypermobility, and/or (6) for moms with obesity.

Reference to connector portion or attachment mechanism herein, such as hook and loop, may additionally include hooks, buttons, snaps, buttons, and/or any other suitable type connector/attachment mechanism and/or technique.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

The terms “comprise,” “comprises,” “comprising,” “include,” “includes,” and “including,” are intended to be non-limiting, such that recitation of items in a list is not to the exclusion of other like items that may be substituted or added to the listed items.

Terms like “preferably,” “commonly,” and “typically” are not utilized herein to limit the scope of the claimed embodiments or to imply that certain features are critical or essential to the structure or function of the claimed embodiments. These terms are intended to highlight alternative or additional features that may or may not be utilized in a particular embodiment of the present invention.

The term “substantially” is utilized herein to represent the inherent degree of uncertainty that may be attributed to any quantitative comparison, value, measurement, or other representation and to represent the degree by which a quantitative representation may vary from a stated reference without resulting in a change in the basic function of the subject matter at issue.

Various modifications and variations of the disclosed methods, compositions and uses of the invention will be apparent to the skilled person without departing from the scope and spirit of the invention. Although the subject matter has been disclosed in connection with specific preferred aspects or embodiments, it should be understood that the subject matter as claimed should not be unduly limited to such specific aspects or embodiments.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments±100%, in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A lumbar support system comprising:
a. an orthotic support band, comprising:
i. a central portion;
ii. a first side portion extending from a first side of the central portion;
iii. a second side portion extending from a second side of the central portion;
iv. a first connector portion disposed on either of an inner surface or an outer surface of an end portion of either of the first side portion or the second side portion and a second connector portion disposed on the other of the inner surface or the outer surface of an end portion of the other of the first side portion or the second side portion, wherein the first connector portion and the second connector portion are configured to engage with one another to secure the first side portion and the second side portion together; and
v. an adjustable pull strap attached to the orthotic support band, and configured to allow for adjusting a fit of the orthotic support band about a user; and
b. one or more of a posterior support panel, anterior support panel, and a pelvic strap.

2. The lumbar support system of claim 1, wherein the central portion further comprises a posterior support panel attachment, wherein the posterior support panel attachment is configured to secure the posterior support panel at the central portion.

3. The lumbar support system of claim 2, wherein the posterior support panel attachment comprises a pouch configured to receive the posterior support panel therein.

4. The lumbar support system of claim 3, wherein the pouch is disposed on either of an exterior or interior surface of the central portion of the orthotic support band.

5. The lumbar support system of claim 1, wherein the anterior support panel is configured to be attachable to either of the first side portion or the second side portion of the orthotic support band.

6. The lumbar support system of claim 1, wherein the anterior support panel is configured to be attachable to an inside surface of either of the first side portion or the second side portion, such that when the orthotic support band is attached and secured about the user the anterior support panel is directly adjacent an anterior portion of the user.

7. The lumbar support system of claim 1, wherein the pelvic strap is configured to extend from the central portion of the orthotic support band to either of the first side portion or the second side portion of the orthotic support band when the first side portion and the second side portion are secured together.

8. The lumbar support system of claim 7, wherein the pelvic strap comprises a posterior end having a first attachment mechanism disposed thereon and an anterior end having one or more finger straps extending therefrom, and wherein the one or more finger straps each include a second attachment mechanism at their distal ends.

9. The lumbar support system of claim 8, wherein the posterior end of the pelvic strap is configured to be attachable to an inside surface of the central portion via the first attachment mechanism, and the anterior end of the pelvic strap is configured to be attachable to an outside surface of either of the first side portion or the second side portion via the second attachment mechanism.

10. The lumbar support system of claim 7, further comprising a cold/hot pack holder, wherein the cold/hot pack holder is configured to attach to the pelvic strap at about a mid-point thereof, and is further configured to retain a cold/hot pack.

11. The lumbar support system of claim 1, wherein the adjustable pull strap comprises a first portion and a second portion, wherein the first portion extends from about a middle point of the central portion in a first direction and the second portion extends from about a middle point of the central portion in a second direction opposite of the first direction, and wherein an end portion of either or both of the first portion and second portion are configured to releasably attach to an outer surface of the first side portion or second side portion.

12. A method of using a lumbar support system, the method comprising:
a. providing an orthotic support band, comprising:
i. a central portion;
ii. a first side portion extending from a first side of the central portion;
iii. a second side portion extending from a second side of the central portion;
iv. a first connector portion disposed on either of an inner surface or an outer surface of an end portion of either of the first side portion or the second side portion and a second connector portion disposed on the other of the inner surface or the outer surface of an end portion of the other of the first side portion or the second side portion, wherein the first connector portion and the second connector portion are configured to engage with one another to secure the first side portion and the second side portion together; and
v. an adjustable pull strap attached to the orthotic support band, and configured to allow for adjusting a fit of the orthotic support band about a user;
b. providing one or more of a posterior support panel, anterior support panel, and a pelvic strap;
c. optionally, attaching one or both of the posterior support panel and the anterior support panel to the orthotic support band;
d. positioning and securing the orthotic support band about a user's body;
e. optionally, adjusting the adjustable pull strap to a desired fit about the user's body; and
f. optionally, attaching the pelvic strap to the orthotic support band.

13. The method of claim 11, further comprising attaching a cold/hot pack holder to the pelvic strap, and attaching or inserting a cold/hot pack to or in the cold/hot pack holder.

14. The method of claim 11, wherein the central portion of the orthotic support band further comprises a posterior support panel attachment, wherein the posterior support panel attachment is configured to secure the posterior support panel at the central portion of the orthotic support band.

15. The method of claim 13, wherein the posterior support panel attachment comprises a pouch configured to receive the posterior support panel therein.

16. The method of claim 14, wherein the pouch is disposed on either of an exterior or interior surface of the central portion of the orthotic support band.

17. The method of claim 11, wherein the anterior support panel is configured to be attachable to either of the first side portion or the second side portion of the orthotic support band.

18. The method of claim 16, wherein the anterior support panel is configured to be attachable to an inside surface of either of the first side portion or the second side portion of the orthotic support band, such that when the orthotic support band is attached and secured about the user the anterior support panel is directly adjacent an anterior portion of the user.

19. The method of claim 11, wherein the pelvic strap is configured to extend from the central portion of the orthotic support band to either of the first side portion or the second side portion of the orthotic support band when the first side portion and the second side portion are secured together.

20. The method of claim 11, wherein the pelvic strap comprises a posterior end having a first attachment mechanism disposed thereon and an anterior end having one or more finger straps extending therefrom, and wherein the one or more finger straps each include a second attachment mechanism at their distal ends.

21. The method of claim 11, wherein the posterior end of the pelvic strap is configured to be attachable to an inside surface of the central portion of the orthotic support band via the first attachment mechanism, and the anterior end of the pelvic strap is configured to be attachable to an outside surface of either of the first side portion or the second side portion of the orthotic support band via the second attachment mechanism.

22. The method of claim 12, wherein the cold/hot pack holder is configured to attach to the pelvic strap at about a mid-point thereof, and is further configured to retain a cold/hot pack.
